(19) United States
(12) Patent Application Publication
(10) Pub. No.: US 2023/0335280 A1
(43) Pub. Date: Oct. 19, 2023

(54) MEDICAL CARE STANDARD KNOWLEDGE-BASED DECISION SUPPORT SYSTEM

(71) Applicant: Kuei-Fang HO, New Taipei City (TW)

(72) Inventor: Kuei-Fang HO, New Taipei City (TW)

(21) Appl. No.: 18/093,278

(22) Filed: Jan. 4, 2023

(30) Foreign Application Priority Data
Apr. 13, 2022 (TW) 111113992

(51) Int. Cl. G16H 50/20; G16H 70/20

(52) U.S. Cl. CPC G16H 50/20; G16H 70/20

(57) ABSTRACT

Disclosed is a medical care standard knowledge-based decision support system that provides clinical decision-making information to a user group including medical-care-related staff in the care of patients, the medical care standard knowledge-based decision support system comprises: a client database; a master knowledge-based relational database; a console; and a medical-care-knowledge-guidance-indicators-constructing-storing device. The medical-care-knowledge-guidance-indicators-constructing-storing device includes a guidance-indicator-list-generating unit, which is configured to receive option judgment results, a knowledge-database-inference-engine is used to generate a plurality of values of weighted ratio and then calculate data of weighted ratio to generate a new guidance-indicator list, the knowledge-database-inference-engine is selected from a Clinical Diagnostic Validity model and a Bayesian decision model.

### FIELD OF THE INVENTION

The present invention relates to a decision support system, and more particularly relates to a medical care standard knowledge-based decision support system.

### BACKGROUND OF THE INVENTION

At present, all domestic nursing professions agrees on the necessity of structural processes in nursing practicing, but there is no consensus on the content in the processes. As a result, each medical institution develops its own practice, resulting in a lack of systematization and standardization in many medical specialties.

Most of the nursing staffs develop individualized patient care plans by directly comparing the collected data with a diagnosis without inspecting the data collected. The defining characteristics and related factors or risk factors of nursing diagnosis are mainly discussed by nursing experts, and most of them still need to be verified by clinical performance. As a result, many clinical nurses are unable to distinguish the importance of each item in their practice.

In psychiatric nursing, for example, the nursing process of clinical nurses can be divided into: establishment of data collection relationships, nursing assessment, establishment of nursing diagnosis, and planning of nursing outcomes and interventions. In terms of process steps, since an item of defining characteristics usually cannot be fully integrated and presented only in a single form or on a single page of an information system, the actual techniques currently used by nursing staff are classified according to the nursing label based on personal knowledge. For many years, in order to make the practice smooth, the nursing staff's operation procedure is to: directly select a nursing diagnosis shown in a displayed form or shown in an information system list after a nursing assessment; and then, based on the subjective and objective data obtained from the assessment, select the defining characteristics and related factors or risk factors that conform to the nursing diagnosis, and further plan nursing outcomes and nursing interventions. However, the decision-making wisdom accumulated by individuals in many patients caring can only be stored in individual's mind, and cannot be fully presented in a form or by an information system. According to the competent thinking process of nursing staff, after completing the assessment, the professionals will follow the clues obtained and directly search the knowledge that has been established through learning and experience in the nurse's mind to determine what are the defining characteristics that meet the assessment results, and whether the number of the defining characteristics meets the requirements for establishing a nursing diagnosis, and then select the related factors by the proposed nursing diagnosis; or complete the established nursing diagnosis by determining what are the risk factors that meet the assessment results, and whether the number of the risk factors meet the requirements for establishing a nursing diagnosis. After completing the establishment of the nursing diagnosis process, the nursing staff will then plan individual nursing outcomes and nursing interventions based on the content of personal wisdom decision-making to achieve the goal of providing appropriate patient care. With the above-mentioned prior arts, if the individual does not have sufficient knowledge, errors are likely to be made when making decisions and judgments, or the selection of items in each step will be found to be insufficient and result in unnecessarily repeated operations, etc., and even worse, the patient's health will be seriously affected due to wrong decision-making.

Therefore, it is still necessary to improve the process of constructing patient care plans and even the decision-making process for various medical care practices.

### SUMMARY OF THE INVENTION

Therefore, one objective of the present invention is to provide a medical care standard knowledge-based decision support system, which can support clinical medical care staff to make clinical decisions with high sensitivity and high specificity in the process of medical care.

In order to overcome the technical problems in prior art, the present invention provides a medical care standard knowledge-based decision support system that provides clinical decision-making information to a user group including medical-care-related staff in the care of patients, the medical care standard knowledge-based decision support system comprises: a client database, which stores basic patient information and medical care process recording information, the medical care process recording information being data which records all clinical decisions made by the user group in the process of medical caring for patients; a master knowledge-based relational database, which provides original content of each one of a plurality of guidance-indicator options to a guidance-indicator-list-generating unit; a console, which is connected to the client database and the master knowledge-based relational database, the console being provided with a user operation interface through which the user group operates the medical care standard knowledge-based decision support system, the console being configured to obtain a guidance-indicator list having the original contents of the guidance-indicator options from a guidance-indicator-list-storing unit and transmit the original contents of the guidance-indicator list to the user operation interface, the user operation interface being provided to receive an option-judgment result corresponding to each of the guidance-indicator options by a user and transmit the option judgment result to the console, and then the option judgment results being transmitted to the guidance-indicator-list-generating unit of a medical-care-knowledge-guidance-indicators-constructing-storing device; and the medical-care-knowledge-guidance-indicators-constructing-storing device, which is connected to the client database, the master knowledge-based relational database and the console, the medical-care-knowledge-guidance-indicators-constructing-storing device including the guidance-indicator-list-generating unit and the guidance-indicator-list-storing unit, the guidance-indicator-list-generating unit being configured to receive the option-judgment results from the console, wherein a knowledge-database-inference-engine is provided to generate a plurality of values of weighted ratio corresponding to the original contents of the respective guidance-indicator options, the guidance-indicator-list-generating unit then calculating data of weighted ratio according to the values of weighted ratio, updating the values of weighted ratio corresponding to the respective guidance-indicator options according to the data of weighted ratio, generating a new guidance-indicator list from the guidance-indicator list by using the updated values of weighted ratio, and storing the new guidance-indicator list in the guidance-indicator-list-storing unit, wherein the console provides the new guidance-indicator list as the guidance-indicator list to the user operation interface such that the original content of each guidance-indicator options in the guidance-indicator list is displayed with the updated values of weighted ratio, and wherein the knowledge-database-inference-engine is selected from a clinical diagnosis validity (CDV) model or a Bayesian decision model.

In one embodiment of the present invention, the master knowledge-based relational database includes a specialized medical record database, a standard nursing assessment database, a standard nursing diagnosis database, a standard nursing outcomes classification database, and a standard nursing interventions classification database, wherein the standard nursing diagnosis database is an international standard nursing language nursing diagnosis database, the standard nursing outcomes classification database is an international standard nursing language nursing outcomes database, and the standard nursing interventions classification database is an international standard nursing language nursing interventions database.

In one embodiment of the present invention, the guidance-indicator list includes: a patient symptoms and signs test and examination guidance-indicator list corresponding to the specialized medical record database; a standard nursing assessment guidance-indicator list corresponding to the standard nursing assessment database; a defining characteristic or risk factors guidance-indicator list, a standard nursing diagnosis guidance-indicator list and a related factors guidance-indicator list corresponding to the standard nursing diagnosis database; a standard nursing outcomes guidance-indicator list corresponding to the standard nursing outcomes classification database; and a standard nursing interventions guidance-indicator list corresponding to the standard nursing interventions classification database.

In one embodiment of the present invention, in the process of generating the new guidance-indicator list by the guidance-indicator-list-generating unit, the guidance-indicator-list-generating unit selects two users in the user group from the client database, and combine outcomes of the two users' plans and care for patients into an item group, the guidance-indicator-list-generating unit sorts out the option-judgment results in correspondence to each item in the item group, and generates the values of weighted ratio by the knowledge-database-inference-engine to calculate the data of weighted ratio.

In one embodiment of the present invention, the clinical diagnosis validity (CDV) model verifies importance of the guidance-indicator options by using the following formula:

R=[A/(A+D)]×[(F1/N+F2/N)/2],

wherein “A” is the number of items having the same state in the option judgment results of the two users; “D” is the number of items having different states in the option judgment results of the two users; “N” is the total number of items in patient group that the two users are jointly responsible for care; “F1” is the total number of items described in the original content of the guidance-indicator options that a first user of the two users has observed; “F2” is the total number of items described in the original content of the guidance-indicator options that a second user of the two users has observed; and “R” is the value of weighted ratio, wherein the guidance-indicator-list-generating unit generates the data of weighted ratio by averaging the values of weighted ratio according to the following formula:

$$W = \frac{{\sum }_{i = 1}^{n} Ri}{n} ,$$

wherein “W” is the data of weighted ratio; “Ri” is each of the values of weighted ratio; and “n” is the total number of the values of weighted ratio.

With the technical means adopted by the medical care standard knowledge-based decision support system of the present invention, after the clinical medical care staffs (for example, clinical nursing staffs) enter the nursing diagnosis stage from the nursing assessment stage, the medical care standard knowledge-based decision support system of the present invention will generate guidance-indicator lists with guidance-indicator options. The guidance-indicator options include defining characteristics, related factors, risk factors and nursing outcomes and nursing interventions to be planned, wherein the defining characteristics, the related factors and the risk factors is formed according to severity, adequacy, and urgency. Accordingly, the clinical medical care staff can refer to the selection during the nursing process. Therefore, the clinical medical care staff can understand the health problems of the patient immediately, and the medical care standard knowledge-based decision support system of the present invention can further assist the clinical medical care staff in making clinical decisions with high sensitivity and high specificity.

### BRIEF DESCRIPTION OF THE DRAWINGS

FIG. 1 is a schematic block view illustrating a medical care standard knowledge-based decision support system according to one embodiment of the present invention;

FIG. 2 is a schematic view illustrating option judgment results of the medical care standard knowledge-based decision support system according to the embodiment of the present invention;

FIG. 3a is a schematic view illustrating a user operation interface of the medical care standard knowledge-based decision support system according to the embodiment of the present invention;

FIG. 3b is a schematic view illustrating a standard nursing assessment guidance-indicator list of the medical care standard knowledge-based decision support system according to the embodiment of the present invention;

FIG. 3c is a schematic view illustrating a defining characteristic or risk factors guidance-indicator list of the medical care standard knowledge-based decision support system according to the embodiment of the present invention;

FIG. 3d is a schematic view illustrating a standard nursing diagnosis guidance-indicator list of the medical care standard knowledge-based decision support system according to the embodiment of the present invention;

FIG. 3e is a schematic view illustrating a related factors guidance-indicator list of the medical care standard knowledge-based decision support system according to the embodiment of the present invention;

FIG. 3f is a schematic view illustrating a standard nursing outcomes guidance-indicator list of the medical care standard knowledge-based decision support system according to the embodiment of the present invention; and

FIG. 3g is a schematic view illustrating a standard nursing interventions guidance-indicator list of the medical care standard knowledge-based decision support system according to the embodiment of the present invention.

### DETAILED DESCRIPTION OF THE PREFERRED EMBODIMENTS

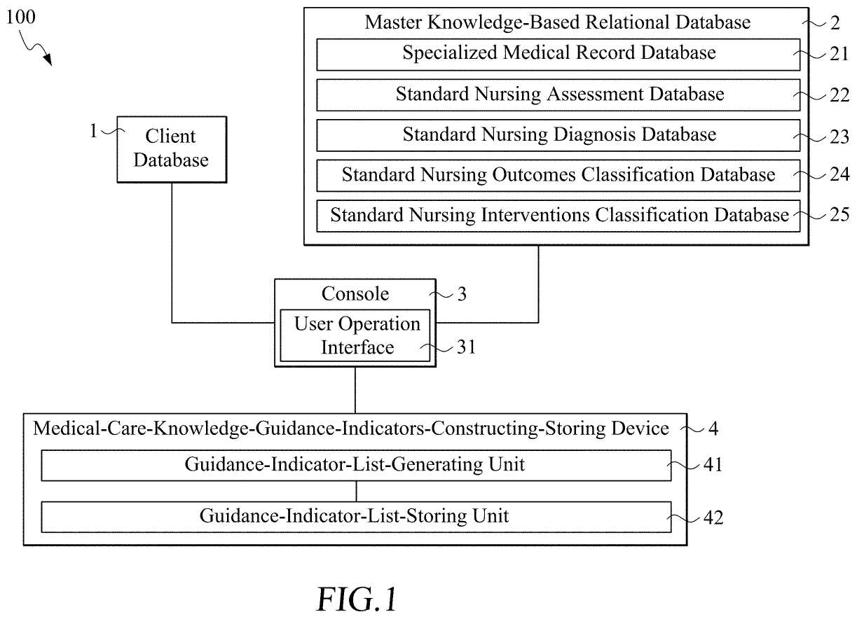

The preferred embodiments of the present invention are described in detail below with reference to FIG. 1 to FIG. 3g. The description is used for explaining the embodiments of the present invention only, but not for limiting the scope of the claims.

As shown in FIG. 1, a medical care standard knowledge-based decision support system 100 that provides clinical decision-making information to a user group including medical-care-related staff in the care of patients according to one embodiment of the present invention comprises: a client database 1, which stores basic patient information and medical care process recording information, the medical care process recording information being data which records all clinical decisions made by the user group in the process of medical caring for patients.

Specifically, the client database 1 is used to store the basic patient information including the patient's admission type, language used, main contact person, etc. The medical care process recording information is a detailed record of the user group who was responsible for caring for the patient, and all records related to the patient's health status during the hospital stay.

As shown in FIG. 1, the medical care standard knowledge-based decision support system 100 according to the embodiment of the present invention comprises a master knowledge-based relational database 2 including a specialized medical record database 21, a standard nursing assessment database 22, a standard nursing diagnosis database 23, a standard nursing outcomes classification database 24, and a standard nursing interventions classification database 25, and the master knowledge-based relational database 2 provides original content of each one of a plurality of guidance-indicator options to a guidance-indicator-list-generating unit 41.

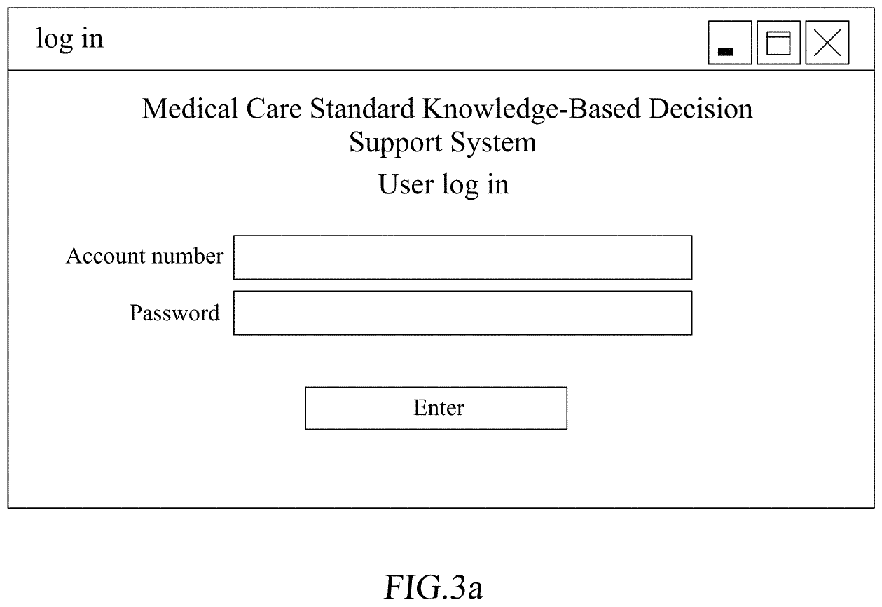

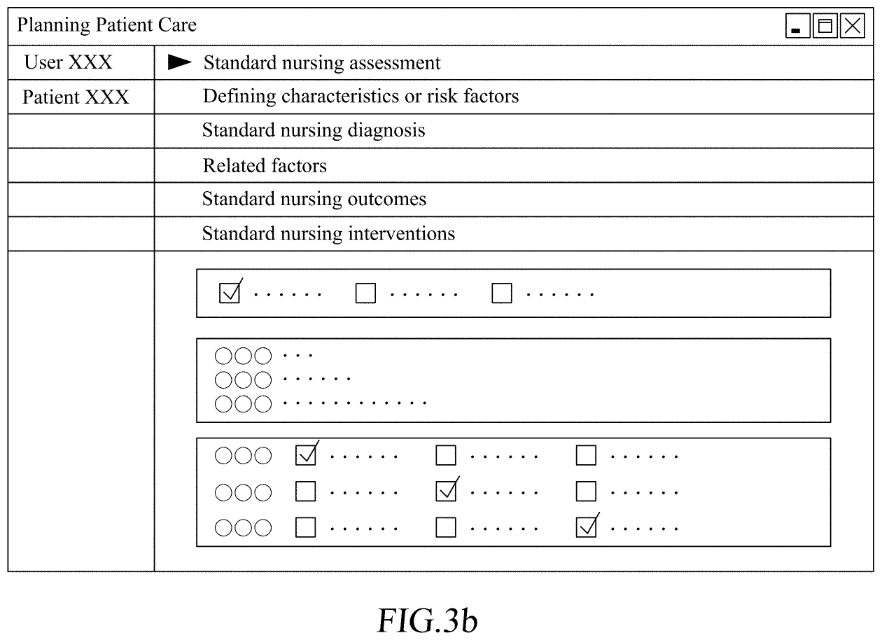

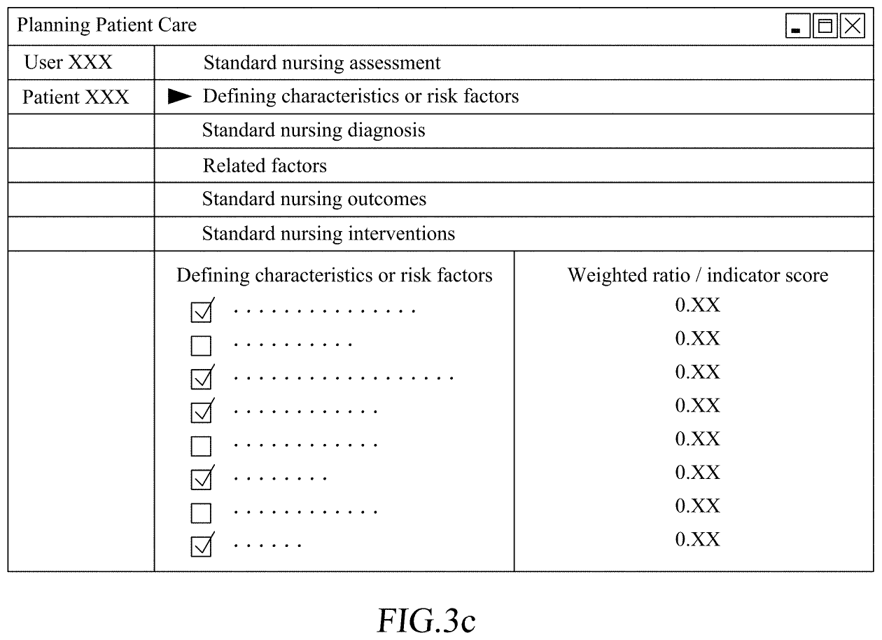

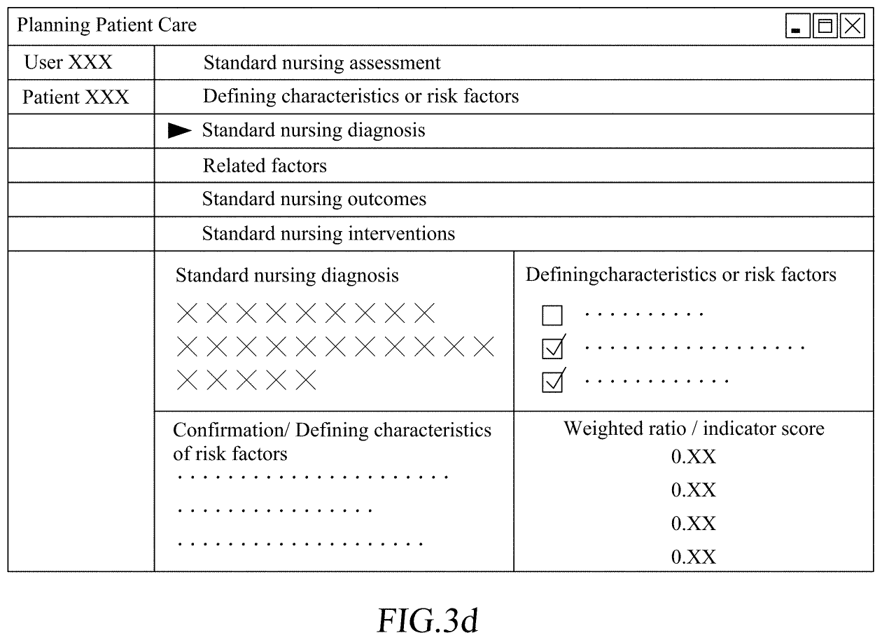

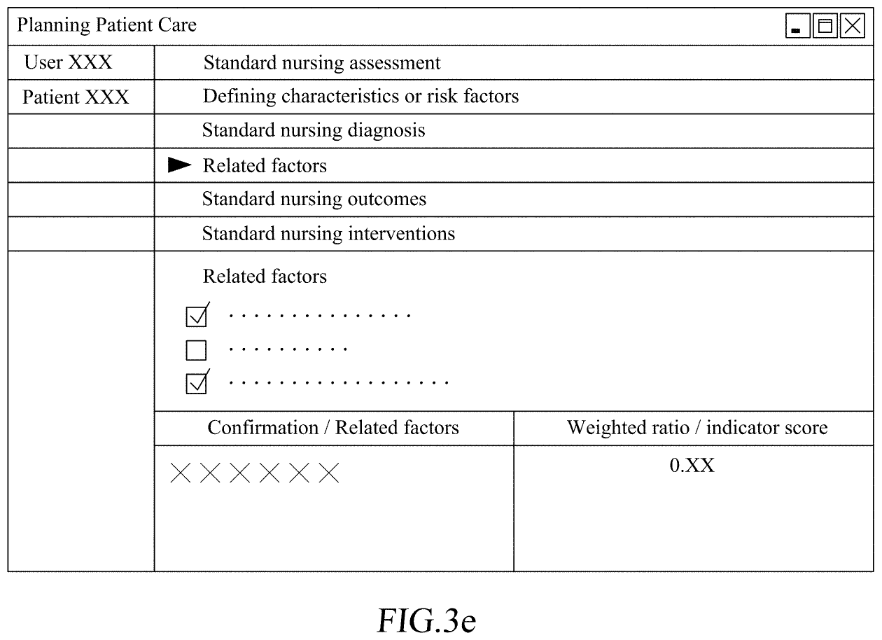

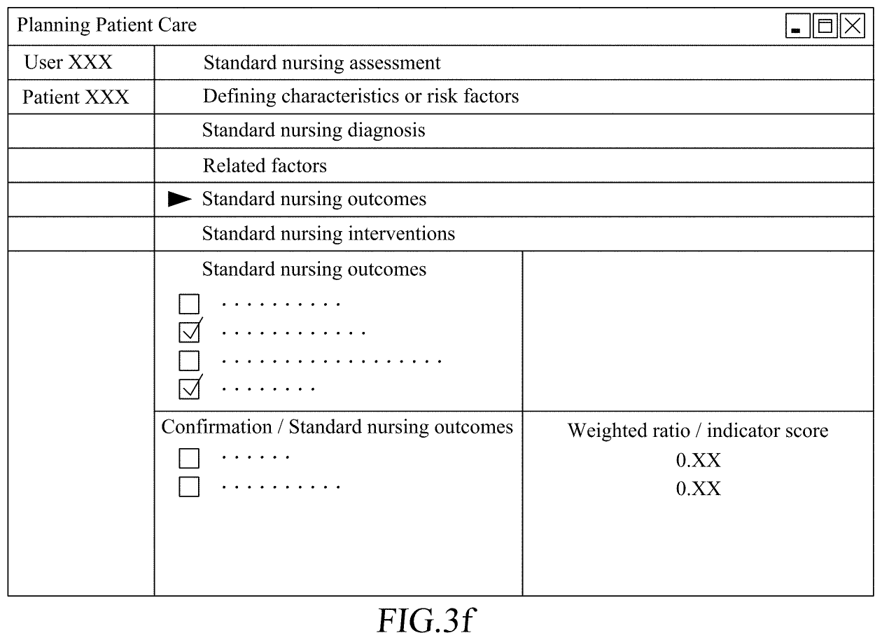

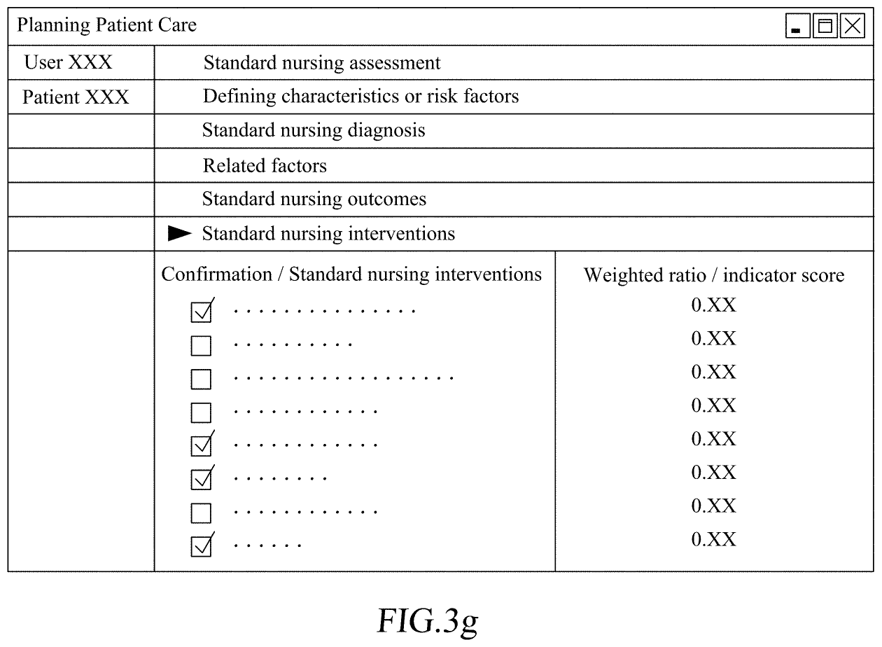

Specifically, the specialized medical record database 21 is a patient's signs, symptoms, lab test, and image examination database; the standard nursing assessment database 22 is an international standard nursing language nursing assessment database, for example, Psychiatric Mental Health Nurses Association standard nursing assessment database; the standard nursing diagnosis database 23 is an international standard nursing language nursing diagnosis database, for example, NANDA (North American Nursing Diagnosis Association) standard nursing diagnosis database; the standard nursing outcomes classification database 24 is an international standard nursing language nursing outcomes database, for example, NOC (Nursing Outcomes Classification) standard nursing outcomes classification database; and the standard nursing interventions classification database 25 is a an international standard nursing language nursing interventions database, for example, NIC (Nursing Interventions Classification) standard nursing interventions classification database. As shown in FIG. 3b, since the embodiment of the present invention takes the nursing process of psychiatric nursing as an example, the specialized medical record database 21 and the standard nursing assessment database 22 in the embodiment are based on the “Five aspects of nursing assessment” recognized by the Psychiatric Mental Health Nursing Association in Taiwan. The medical care standard knowledge-based decision support system 100 of the present invention is not limited to this, and according to different medical specialties, any specialized medical record database and a standard assessment database recognized by such medical specialties professions can also be used as the specialized medical record database 21 and the standard nursing assessment database 22.

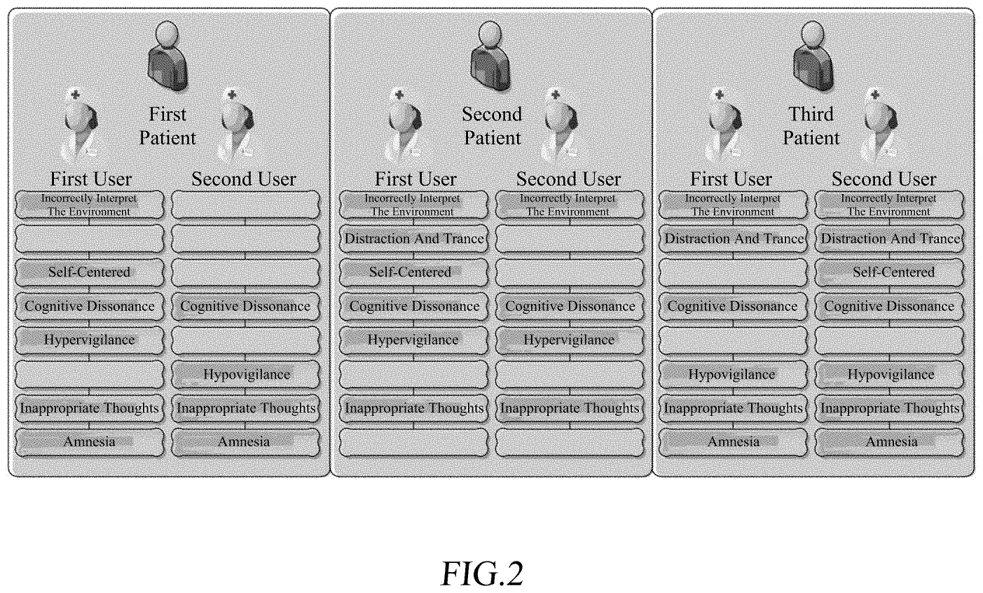

As shown in FIG. 1 and FIG. 3a to FIG. 3g, the medical care standard knowledge-based decision support system 100 comprises a console 3, which is connected to the client database 1 and the master knowledge-based relational database 2, the console 3 is provided with a user operation interface 31 through which the user group operates the medical care standard knowledge-based decision support system 100, the console 3 is configured to obtain a guidance-indicator list having the original contents of the guidance-indicator options from a guidance-indicator-list-storing unit 42 and transmit the original contents of the guidance-indicator list to the user operation interface 31, the user operation interface 31 is provided to receive an option judgment result corresponding to each of the guidance-indicator options by a user (for example, FIG. 2 shows each of the option-judgment results selected by two users for each of the guidance-indicator options (i.e., defining characteristics) in the defining characteristics guidance-indicator list. Taking a first patient, who is jointly cared for by a first user and a second user, as an example, the first user considers that the first patient has the defining characteristic of “incorrectly interpret the environment” after observing the first patient, so the first user then ticks this guidance-indicator option in the user operation interface 31, meanwhile, the result of “ticking the guidance-indicator option” is “the option-judgment result”; on the other hand, the second user considers that the first patient does not have the defining characteristic of “incorrectly interpret the environment” after observing the first patient, so the second user does not tick this guidance-indicator option in the user operation interface 31, meanwhile, the result of “the guidance-indicator option is not ticked” is “the option judgment result”. In summary, the option judgment results for the two users are in different states.) and transmit the option judgment result to the console 3, and then the option judgment results are transmitted to a guidance-indicator-list-generating unit 41 of a medical-care-knowledge-guidance-indicators-constructing-storing device 4.

Specifically, as shown in FIG. 3b, the guidance-indicator list includes a standard nursing assessment guidance-indicator list corresponding to the standard nursing assessment database 22; as shown in FIG. 3c, the guidance-indicator list includes a defining characteristic or risk factors guidance-indicator list corresponding to the standard nursing diagnosis database 23; as shown in FIG. 3d, the guidance-indicator list includes a standard nursing diagnosis guidance-indicator list corresponding to the standard nursing diagnosis database 23; as shown in FIG. 3e, the guidance-indicator list includes a related factors guidance-indicator list corresponding to the standard nursing diagnosis database 23; as shown in FIG. 3f, the guidance-indicator list includes a standard nursing outcomes guidance-indicator list corresponding to the standard nursing outcomes classification database 24; and as shown in FIG. 3g, the guidance-indicator list includes a standard nursing interventions guidance-indicator list corresponding to the standard nursing interventions classification database 25. Each of the databases included in the master knowledge-based relational database 2 can generate the corresponding guidance-indicator list.

As shown in FIG. 1, the medical care standard knowledge-based decision support system 100 according to the embodiment of the present invention comprises the medical-care-knowledge-guidance-indicators-constructing-storing device 4, which is connected to the client database 1, the master knowledge-based relational database 2 and the console 3, the medical-care-knowledge-guidance-indicators-constructing-storing device 4 includes the guidance-indicator-list-generating unit 41 and the guidance-indicator-list-storing unit 42, the guidance-indicator-list-generating unit 41 is configured to receive the option judgment results from the console 3, wherein a knowledge-database-inference-engine is provided to generate a plurality of values of weighted ratio (or indicator score) corresponding to the original contents of the respective guidance-indicator options, the guidance-indicator-list-generating unit 41 then calculates data of weighted ratio (or indicator score) according to the values of weighted ratio (or indicator score), updates the values of weighted ratio (or indicator score) corresponding to the respective guidance-indicator options according to the data of weighted ratio (or indicator score), generates a new guidance-indicator list from the guidance-indicator list by using the updated values of weighted ratio (or indicator score), and stores the new guidance-indicator list in the guidance-indicator-list-storing unit 42, wherein the console 3 provides the new guidance-indicator list as the guidance-indicator list to the user operation interface 31 such that the original content of each guidance-indicator options in the guidance-indicator list is displayed with the updated values of weighted ratio (or indicator score) (as shown in FIG. 3b to FIG. 3g), the knowledge-database-inference-engine is selected from a clinical diagnosis validity (CDV) model or a Bayesian decision model. With the spread of the master knowledge-based relational database 2, the medical-care-knowledge-guidance-indicators-constructing-storing device 4 can be expanded and more accurate by controlling it to support nursing staff in making autonomous decisions about patient care plans.

As shown in FIG. 1 to FIG. 2, in the medical care standard knowledge-based decision support system 100 according to the embodiment of the present invention, in the process of generating the new guidance-indicator list by the guidance-indicator-list-generating unit 41, the guidance-indicator-list-generating unit 41 selects two users in the user group from the client database, one is an advanced clinical nursing staff and the other is a clinical general nursing staff, and combines outcomes of the two users' plans and care for patients into an item group, the guidance-indicator-list-generating unit 41 sorts out the option judgment results in correspondence to each item in the item group, and generates the values of weighted ratio (or indicator score) by the knowledge-database-inference-engine to calculate the data of weighted ratio (or indicator score).

According to the medical care standard knowledge-based decision support system 100 in the embodiment of the present invention, the clinical diagnosis validity (CDV) model (Fehring, 1987) is based on clinical decision results made by one advanced clinical nursing staff and one clinical general nursing staff, the CDV model verifies importance of the guidance-indicator options by using the following formula:

R=[A/(A+D)]×[(F1/N+F2/N)/2],
wherein “A” is the number of items having the same state in the option judgment results of the two users;
“D” is the number of items having different states in the option judgment results of the two users;
“N” is the total number of items in patient group that the two users are jointly responsible for care;
“F1” is the total number of items described in the original content of the guidance-indicator options that a first user of the two users has observes;
“F2” is the total number of items described in the original content of the guidance-indicator options that a second user of the two users has observes; and
“R” is the value of weighted ratio,
wherein the guidance-indicator-list-generating unit generates the data of weighted ratio by averaging the values of weighted ratio according to the following formula:

$$W = \frac{{\sum }_{i = 1}^{n} Ri}{n} ,$$

wherein “W” is the data of weighted ratio;
“Ri” is each of the values of weighted ratio; and
“n” is the total number of the values of weighted ratio.

In the embodiment, the combination of the two users is selected from: a combination of one practical nursing specialist (e.g., the advanced clinical nursing staff) and one clinical general nursing staff, a combination of two practical nursing specialists, and a combination of two clinical general nursing staffs.

Specifically, as shown in FIG. 2, the total number of items in the item group that the first user and the second user are jointly responsible for care is 3 patients (“N” is 3), which are represented by a first patient, a second patient and a third patient, respectively. Taking the guidance-indicator option of “distraction and trance” as an example, the number of items having the same state in the option judgment results of the two users is 2 patients (i.e., “A” is 2, which are the first patient and the third patient respectively), the number of items having different states in the option judgment results of the two users is 1 patient (i.e., “D” is 1, which is the second patient), the total number of items described in the guidance-indicator option of “distraction and trance” that the first user of the two users has observes is 2 patients (i.e., “F1” is 2, which are the second patient and the third patient), the total number of items described in the guidance-indicator option of “distraction and trance” that the second user of the two users has observes is 1 patient (i.e., “F2” is 1, which is the third patient). Therefore, the value of weighted ratio R is [2/2+1]×[(2/3+1/3)/2]=0.33.

Furthermore, after the guidance-indicator-list-generating unit 41 calculates the data of weighted ratio W according to a plurality of the values of weighted ratio Ri, updates the values of weighted ratio corresponding to the respective guidance-indicator options according to the data of weighted ratio W, and generates a new guidance-indicator list from the guidance-indicator list by using the updated values of weighted ratio, when the value of weighted ratio>0.8, it indicates that the importance degree of the guidance-indicator option is a critical or major characteristic; when the value of weighted ratio>0.5 and<0.8, it indicates that the importance degree of the guidance-indicator option is a relevant or minor characteristic; and when the value of weighted ratio<0.5, it indicates that the importance degree of the guidance-indicator option should be removed.

Specifically, when the user group uses the medical care standard knowledge-based decision support system 100 of the present invention in the nursing process, while making clinical decisions, in addition to using the user group's own medical knowledge and experience, it is also possible to refer to each of the values of weighted ratio displayed in each of the guidance-indicator options on each of the guidance-indicator lists. The medical care standard knowledge-based decision support system 100 of the present invention can thereby support the clinical medical care staff to make clinical decisions with high sensitivity and high specificity.

In addition, taking the nursing process as an example, the steps of the nursing process are: nursing assessment, nursing diagnosis (including from observing and judging “defining characteristics” to observing and judging “related factors”; or observing and judging “risk factors”), nursing outcomes and nursing interventions. When the user group progresses from one step (e.g., observing and judging “defining characteristics” or “risk factors”) in the nursing process to the next (e.g., observing and judging “related factors”), and must make corresponding decisions, the user group makes clinical decisions with high sensitivity and specificity by selecting each of the guidance-indicator options corresponding to the value of weighted ratio (or indicator score) that has been calculated by the knowledge-database-inference-engine. In other words, when the user group executes the medical care behavior process through the medical care standard knowledge-based decision support system 100 of the present invention, the value of weighted ratio (or indicator score) calculated by the knowledge-database-inference-engine (selected from the CDV model or the Bayesian decision model) must be referenced between each step of the process, thereby making a decision.

When the knowledge-database-inference-engine is the Bayesian decision model, the medical care standard knowledge-based decision support system 100 of the present invention obtains a decision-guidance index by calculating the association relationship between the nursing assessment and the defining characteristics and related factors or risk factors in a clinical practice collection database through a Bayesian formula, wherein the Bayesian formula is as follows:

$$P ( DC \vert + ) = \frac{P ( + \vert DC ) P ( DC )}{P ( + \vert DC ) P ( DC ) + P ( + \vert Non\_DC ) P ( Non\_DC )} ,$$

wherein “DC” is a defining characteristic item which is actually occurred;
“+” is a nursing assessment item which is actually occurred;
“Non_DC” is the defining characteristic item which is not actually occurred;
“P(+|DC)” is a conditional probability of observing the nursing assessment item and having identified a specific defining characteristic;
“P(DC)” is a marginal probability of observing the presence of the specific defining characteristic in all patients;
“P(+|Non_DC)” is a conditional probability of observing the nursing assessment item without identifying the specific defining characteristic;
“P(Non_DC)” is a marginal probability of not observing the presence of the specific defining characteristic in all patients; and
“P(DC|+)” is a likelihood value that the specific defining characteristic has been identified and that a nursing assessment item does exist.

“Bayesian decision model” is to calculate the probabilistic relationship between diseases and symptoms by using in a conditional probability manner with high-quality datasets compiled in the clinical medical environment, so as to provide information-based decision-making assistance guidance (Cypko & Stoehr, 2019; Kumar, 2017; Liu, Lu, Ma, Chen, & Qin, 2016; M. Xu & Shen, 2013).

With the technical means adopted by the medical care standard knowledge-based decision support system 100 of the present invention, after the clinical medical care staffs, such as the clinical nursing staffs, enter the nursing diagnosis stage from the nursing assessment stage, the medical care standard knowledge-based decision support system 100 of the present invention will generate guidance-indicator lists with various guidance-indicator options, The guidance-indicator options include defining characteristics, related factors, risk factors and nursing outcomes and nursing interventions to be planned, wherein the defining characteristics, the related factors and the risk factors is formed according to severity, adequacy, and urgency. Accordingly, the clinical nursing staffs can refer to the selection during the nursing process. Therefore, the clinical nursing staffs can understand the health problems of the patient immediately, and the medical care standard knowledge-based decision support system 100 of the present invention can further assist the clinical nursing staffs in making clinical decisions with high sensitivity and high specificity.

In addition, the use of the international standard nursing language nursing diagnosis, the international standard nursing language nursing outcomes, and the international standard nursing language nursing interventions can also ensure that when providing nursing care, the medical care staffs can use the standardized nursing language to provide rich and consistent descriptions of relevant nursing care, and facilitate computerization, simplified records and different types of nursing care, and furthermore, the medical care standard knowledge-based decision support system 100 of the present invention can be made applicable to all situations and fields of clinical specialties presenting medical care.

The medical care standard knowledge-based decision support system 100 of the present invention can better match the definition of the health problem, remove unnecessary items of the related factors or the defining characteristics, distinguish between major and minor items, determine whether they are sufficient to present the defining characteristics of the diagnosis, and provide a good direction for medical care staffs to identify health problems and influencing factors in each case at an early stage.

The above description is only an explanation of the preferred embodiments of the present invention. One having ordinary skill in the art can make various modifications according to the above description and the claims defined below. However, those modifications shall still fall within the scope of the present invention.

What is claimed is:

1. A medical care standard knowledge-based decision support system that provides clinical decision-making information to a user group including medical-care-related staff in the care of patients, the medical care standard knowledge-based decision support system comprises:
a client database, which stores basic patient information and medical care process recording information, the medical care process recording information being data which records all clinical decisions made by the user group in the process of medical caring for patients;
a master knowledge-based relational database, which provides original content of each one of a plurality of guidance-indicator options to a guidance-indicator-list-generating unit;
a console, which is connected to the client database and the master knowledge-based relational database, the console being provided with a user operation interface through which the user group operates the medical care standard knowledge-based decision support system, the console being configured to obtain a guidance-indicator list having the original contents of the guidance-indicator options from a guidance-indicator-list-storing unit and transmit the original contents of the guidance-indicator list to the user operation interface, the user operation interface being provided to receive an option judgment result corresponding to each of the guidance-indicator options by a user and transmit the option judgment result to the console, and then the option judgment results being transmitted to the guidance-indicator-list-generating unit of a medical-care-knowledge-guidance-indicators-constructing-storing device; and
the medical-care-knowledge-guidance-indicators-constructing-storing device, which is connected to the client database, the master knowledge-based relational database and the console, the medical-care-knowledge-guidance-indicators-constructing-storing device including the guidance-indicator-list-generating unit and the guidance-indicator-list-storing unit, the guidance-indicator-list-generating unit being configured to receive the option-judgment results from the console, wherein a knowledge-database-inference-engine is provided to generate a plurality of values of weighted ratio corresponding to the original contents of the respective guidance-indicator options, the guidance-indicator-list-generating unit then calculating data of weighted ratio according to the values of weighted ratio, updating the values of weighted ratio corresponding to the respective guidance-indicator options according to the data of weighted ratio, generating a new guidance-indicator list from the guidance-indicator list by using the updated values of weighted ratio, and storing the new guidance-indicator list in the guidance-indicator-list-storing unit,
wherein the console provides the new guidance-indicator list as the guidance-indicator list to the user operation interface such that the original content of each guidance-indicator options in the guidance-indicator list is displayed with the updated values of weighted ratio, and wherein the knowledge-database-inference-engine is selected from a clinical diagnosis validity (CDV) model or a Bayesian decision model.

2. The medical care standard knowledge-based decision support system as claimed in claim 1, wherein the master knowledge-based relational database includes a specialized medical record database, a standard nursing assessment database, a standard nursing diagnosis database, a standard nursing outcomes classification database, and a standard nursing interventions classification database, wherein the standard nursing diagnosis database is an international standard nursing language nursing diagnosis database, the standard nursing outcomes classification database is an international standard nursing language nursing outcomes database, and the standard nursing interventions classification database is an international standard nursing language nursing interventions database.

3. The medical care standard knowledge-based decision support system as claimed in claim 2, wherein the guidance-indicator list includes: a patient symptoms and signs test and examination guidance-indicator list corresponding to the specialized medical record database; a standard nursing assessment guidance-indicator list corresponding to the standard nursing assessment database; a defining characteristic or risk factors guidance-indicator list, a standard nursing diagnosis guidance-indicator list and a related factors guidance-indicator list corresponding to the standard nursing diagnosis database; a standard nursing outcomes guidance-indicator list corresponding to the standard nursing outcomes classification database; and a standard nursing interventions guidance-indicator list corresponding to the standard nursing interventions classification database.

4. The medical care standard knowledge-based decision support system as claimed in claim 1, wherein in the process of generating the new guidance-indicator list by the guidance-indicator-list-generating unit, the guidance-indicator-list-generating unit selects two users in the user group from the client database, and combine outcomes of the two users' plans and care for patients into an item group, the guidance-indicator-list-generating unit sorts out the option-judgment results in correspondence to each item in the item group, and generates the values of weighted ratio by the knowledge-database-inference-engine to calculate the data of weighted ratio.

5. The medical care standard knowledge-based decision support system as claimed in claim 1, wherein the clinical diagnosis validity (CDV) model verifies importance of the guidance-indicator options by using the following formula:
R=[A/(A+D)]×[(F1/N+F2/N)/2],
wherein “A” is the number of items having the same state in the option judgment results of the two users;
“D” is the number of items having different states in the option judgment results of the two users;
“N” is the total number of items in patient group that the two users are jointly responsible for care;
“F1” is the total number of items described in the original content of the guidance-indicator options that a first user of the two users has observed;
“F2” is the total number of items described in the original content of the guidance-indicator options that a second user of the two users has observed; and
“R” is the value of weighted ratio,
wherein the guidance-indicator-list-generating unit generates the data of weighted ratio by averaging the values of weighted ratio according to the following formula:
$$W = \frac{{\sum }_{i = 1}^{n} Ri}{n} ,$$
wherein “W” is the data of weighted ratio;
“Ri” is each of the values of weighted ratio; and
“n” is the total number of the values of weighted ratio.

6. The medical care standard knowledge-based decision support system as claimed in claim 1, wherein the guidance-indicator list includes: a patient symptoms and signs test and examination guidance-indicator list corresponding to the specialized medical record database; a standard nursing assessment guidance-indicator list corresponding to the standard nursing assessment database; a defining characteristic or risk factors guidance-indicator list, a standard nursing diagnosis guidance-indicator list and a related factors guidance-indicator list corresponding to the standard nursing diagnosis database; a standard nursing outcomes guidance-indicator list corresponding to the standard nursing outcomes classification database; and a standard nursing interventions guidance-indicator list corresponding to the standard nursing interventions classification database.
